(19) United States
(12) Patent Application Publication
(10) Pub. No.: US 2026/0108660 A1
(43) Pub. Date: Apr. 23, 2026

(54) ARTIFICIAL BONE MATERIAL AND METHOD FOR PREPARING THE SAME

(71) Applicant: Taipei Medical University, Taipei City (TW)

(72) Inventors: Jui-Sheng Sun, Taipei City (TW); Yi-Jie Kuo, New Taipei City (TW); Man-Hai Liu, Taipei City (TW); Chih-Hsiang Fang, Taipei City (TW)

(21) Appl. No.: 19/362,644

(22) Filed: Oct. 20, 2025

Related U.S. Application Data
(60) Provisional application No. 63/708,933, filed on Oct. 18, 2024.

(30) Foreign Application Priority Data
Oct. 28, 2024 (TW) 113141070

(51) Int. Cl. A61L 27/46; A61L 27/54; D01D 5/00

(52) U.S. Cl. CPC A61L 27/46; A61L 27/54; D01D 5/0038; A61L 2430/02

(57) ABSTRACT

An artificial bone material includes a natural polymer, an inorganic salt, and Pueraria lobata. The natural polymer includes at least one of gelatin, collagen, hyaluronic acid, alginate, chitosan and their derivatives. The inorganic salt includes at least one of hydroxyapatite (HAP), tricalcium phosphate (TCP), dicalcium phosphate (DCP), dicalcium phosphate dihydrate (DCPD), tetracalcium phosphate (TTCP) and their derivatives. The present invention further provides a method for preparing the artificial bone material, including mixing the natural polymer, the inorganic salt, and Pueraria lobata uniformly, followed by electrospinning and drying to form the artificial bone material. The artificial bone material has the function of promoting stem cell differentiation and bone regeneration.

### CROSS-REFERENCE TO RELATED APPLICATION

The present application claims benefit to U.S. Provisional patent application No. 67/708,933, filed on October 24, 2024, which is hereby incorporated by reference in the entirety.

### FIELD OF THE INVENTION

The present invention relates to the field of medical devices, and more particularly to an artificial bone material used for bone tissue repair, as well as a method for preparing the same.

### BACKGROUND OF THE INVENTION

Artificial bone materials play an important role in various medical applications. In orthopedic surgery, artificial bone materials can promote bone regeneration and repair in cases of bone defects caused by trauma, tumor resection, or infection. For fractures that are difficult to heal, such bone materials can provide essential structural support and thereby facilitate bone healing. In reconstructive procedures, such as craniofacial surgery, artificial bone grafts can be used to fill and reconstruct bone structures. In the dental field, artificial bone materials may be used to augment the alveolar bone when teeth are missing, thereby providing a stable foundation for dental implants. In addition, certain artificial bone materials can be combined with bone marrow transplantation to enhance angiogenesis and promote bone regeneration.

### SUMMARY OF THE INVENTION

The present invention provides an artificial bone material capable of promoting the differentiation of mesenchymal stem cells (MSC) and facilitating bone regeneration.

In order to achieve one, some, or all of the above objects or other objects, the present invention provides an artificial bone material including a natural polymer, an inorganic salt, and Pueraria lobata (kudzu root). The natural polymer includes at least one of gelatin, collagen, hyaluronic acid, alginate, chitosan, and their derivatives. The inorganic salt includes at least one of hydroxyapatite, tricalcium phosphate, dicalcium phosphate, dicalcium phosphate dihydrate, tetracalcium phosphate, and their derivatives.

In one embodiment of the present invention, based on a total weight of the artificial bone material, a weight percentage of the natural polymer is between 45 wt% and 95 wt%, a weight percentage of the inorganic salt is between 1 wt% and 10 wt%, and a weight percentage of Pueraria lobata is between 0.01 wt% and 50 wt%.

According to an embodiment, the present invention provides a method for preparing an artificial bone material. The method includes preparing a bone material solution, an electrospinning process, and a drying process. First, a natural polymer, an inorganic salt, and Pueraria lobata are mixed in sterile water to form the bone material solution. The electrospinning process is then performed on the bone material solution to form a nanofiber scaffold. After that, the drying process is performed to freeze-dry the nanofiber scaffold to remove water and form the artificial bone material.

In one embodiment of the present invention, the bone material solution includes the natural polymer at a concentration of between 0.1 g/L and 1000 g/L, the inorganic salt at a concentration of between 0.1 g/L and 1000 g/L, and the Pueraria lobata at molar concentration of between 1 μM and 5 M.

In one embodiment of the present invention, the bone material solution includes the natural polymer at a concentration of between 1 g/L and 500 g/L, the inorganic salt at a concentration of between 0.5 g/L and 100 g/L, and the Pueraria lobata at molar concentration of between 10 μM and 5 M.

In one embodiment of the present invention, preparing the bone material solution is first dissolving the natural polymer into the sterile water to form a natural polymer solution. The inorganic salt is then added into the natural polymer solution to obtain a mixed solution. After that, the Pueraria lobata is added into the mixed solution to form the bone material solution. During preparing the bone material solution, temperatures of the natural polymer solution, the mixed solution, and the bone material solution are between 35 ℃ and 65 ℃.

In one embodiment of the present invention, the natural polymer includes at least one of gelatin, collagen, hyaluronic acid, alginate, chitosan, and their derivatives. The inorganic salt comprises at least one of hydroxyapatite, tricalcium phosphate, dicalcium phosphate, dicalcium phosphate dihydrate, tetracalcium phosphate, and their derivatives.

In one embodiment of the present invention, the electrospinning process is operated at an ambient temperature of 35 ℃ to 65 ℃ and at a working voltage of 10 kV to 100 kV.

In one embodiment of the present invention, the drying process is performed at a freezing temperature of -196 ℃ to 4 ℃ for a duration of 24 hours to 72 hours.

The present invention provides the artificial bone material including the natural polymer, the inorganic salt, and the Pueraria Lopata for promoting the differentiation of mesenchymal stem cells, thereby facilitating bone regeneration .

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a flow chart of a method for preparing an artificial bone material according to an embodiment of the invention.

FIG. 2 is a flow chart of preparing a bone material solution according to an embodiment of the invention.

FIG. 3 is a schematic diagram showing normalized expression amounts of the ALP gene in an artificial bone material according to a first embodiment of the invention, relative to a control group.

FIG. 4 is a schematic diagram showing normalized expression amounts of the BGLAP gene in the artificial bone material according to the first embodiment of the invention, relative to the control group.

FIG. 5 is a schematic diagram showing normalized expression amounts of the COL1A1 gene in the artificial bone material according to the first embodiment of the invention, relative to the control group.

FIG. 6 is a schematic diagram showing normalized expression amounts of the RUNX2 gene in the artificial bone material according to the first embodiment of the invention, relative to the control group.

FIG. 7 is a schematic diagram showing normalized expression amounts of the SP7 gene in the artificial bone material according to the first embodiment of the invention, relative to the control group.

FIG. 8 is a schematic diagram showing normalized expression amounts of the SPARC gene in the artificial bone material according to the first embodiment of the invention, relative to the control group.

FIG. 9 is a schematic diagram showing normalized expression amounts of the ALP gene in an artificial bone material according to a second embodiment of the invention, relative to the control group.

FIG. 10 is a schematic diagram showing normalized expression amounts of the BGLAP gene in the artificial bone material according to the second embodiment of the invention, relative to the control group.

FIG. 11 is a schematic diagram showing normalized expression amounts of the COL1A1 gene in the artificial bone material according to the second embodiment of the invention, relative to the control group.

FIG. 12 is a schematic diagram showing normalized expression amounts of the RUNX2 gene in the artificial bone material according to the second embodiment of the invention, relative to the control group.

FIG. 13 is a schematic diagram showing normalized expression amounts of the SP7 gene in the artificial bone material according to the second embodiment of the invention, relative to the control group.

FIG. 14 is a schematic diagram showing normalized expression amounts of the SPARC gene in the artificial bone material according to the second embodiment of the invention, relative to the control group.

FIG. 15 is a schematic diagram showing normalized expression amounts of the ALP gene in an artificial bone material according to a third embodiment of the invention, relative to the control group.

FIG. 16 is a schematic diagram showing normalized expression amounts of the BGLAP gene in the artificial bone material according to the third embodiment of the invention, relative to the control group.

FIG. 17 is a schematic diagram showing normalized expression amounts of the COL1A1 gene in the artificial bone material according to the third embodiment of the invention, relative to the control group.

FIG. 18 is a schematic diagram showing normalized expression amounts of the RUNX2 gene in the artificial bone material according to the third embodiment of the invention, relative to the control group.

FIG. 19 is a schematic diagram showing normalized expression amounts of the SP7 gene in the artificial bone material according to the third embodiment of the invention, relative to the control group.

FIG. 20 is a schematic diagram showing normalized expression amounts of the SPARC gene in the artificial bone material according to the third embodiment of the invention, relative to the control group.

### DETAILED DESCRIPTION OF PREFERRED EMBODIMENTS

The present invention provides an artificial bone material including a natural polymer, an inorganic salt, and Pueraria lobata. The natural polymer includes at least one of gelatin, collagen, hyaluronic acid, alginate, chitosan, and their derivatives. The inorganic salt includes at least one of hydroxyapatite, tricalcium phosphate, dicalcium phosphate, dicalcium phosphate dihydrate, tetracalcium phosphate, and their derivatives.

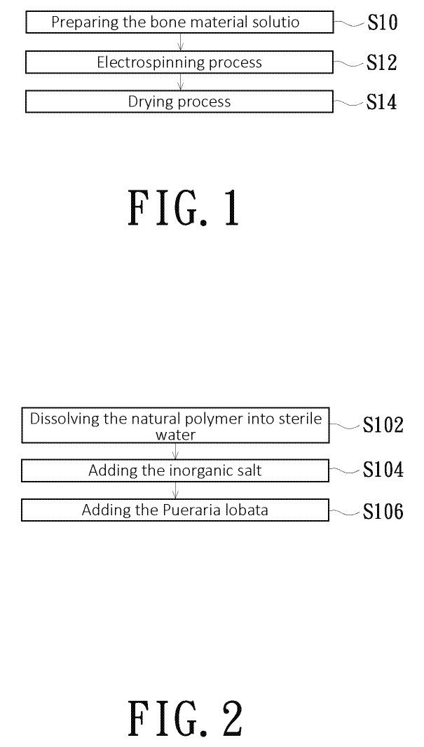

FIG. 1 is a flow chart of a method for preparing an artificial bone material according to an embodiment of the invention. FIG. 2 is a flow chart of preparing a bone material solution according to an embodiment of the invention. As shown in FIG. 1 and FIG. 2, the method for preparing the artificial bone material includes: preparing the bone material solution (Step S10), an electrospinning process (Step S12), and a drying process (Step S14). In addition, step S10 of preparing the bone material solution may further include dissolving the natural polymer into the sterile water to form a natural polymer solution (Step S102), adding the inorganic salt into the natural polymer solution to obtain a mixed solution (Step S104), and adding the Pueraria lobata into the mixed solution to form the bone material solution (Step S106). In Step S10 of preparing the bone material solution, temperatures of the natural polymer solution, the mixed solution, and the bone material solution are between 35 ℃ and 65 ℃.

Furthermore, the bone material solution may include the natural polymer at a concentration of between 0.1 g/L and 1000 g/L, the inorganic salt at a concentration of between 0.1 g/L and 1000 g/L, and the Pueraria lobata at molar concentration of between 1 μM and 5 M. In one embodiment of the present invention, the bone material solution may include the natural polymer at a concentration of between 1 g/L and 500 g/L, the inorganic salt at a concentration of between 0.5 g/L and 100 g/L, and the Pueraria lobata at molar concentration of between 10 μM and 5 M. In another embodiment of the present invention, the bone material solution may include the natural polymer at a concentration of between 50 g/L and 250 g/L, the inorganic salt at a concentration of between 1 g/L and 50 g/L, and the Pueraria lobata at molar concentration of between 50 μM and 5 M.

Specifically, in Step S102, for example, at a temperature of 35 ℃ to 65 ℃, 5 g to 25 g of the natural polymer may be dissolved into the sterile water with continuous stirring until the natural polymer is uniformly dissolved, thereby forming the natural polymer solution. However, the invention is not limited thereto. In Step S104, for example, at a temperature of 35 ℃ to 65 ℃, 0.1 g to 5 g of the inorganic salt may be dissolved into the natural polymer solution with continuous stirring until the inorganic salt is uniformly dissolved, thereby forming the mixed solution. However, the invention is not limited thereto. In Step S106, for example, at a temperature of 35 ℃ to 65 ℃, 0.002 g to 20 g of the Pueraria lobata may be dissolved into the mixed solution with continuous stirring until the Pueraria lobata is uniformly dissolved, thereby forming the bone material solution. However, the invention is not limited thereto. In an embodiment, a time period of continuous stirring to form the natural polymer solution may be between 10 minutes and 300 minutes. A time period of continuous stirring to form the mixed solution may be between one hour and 96 hours. A time period of continuous stirring to form the bone material solution may be between 10 minutes and 300 minutes. However, the invention is not limited thereto.

In Step 12, the electrospinning process includes configuring the operating conditions of an electrospinning apparatus and operating the electrospinning apparatus to perform the electrospinning process on the bone material solution, thereby forming nanofiber scaffolds. In an embodiment, the electrospinning apparatus includes a feeder, a material tube, spinning nozzle, a material collection plate, an electrostatic generator, and a temperature controller. The feeder contains a raw material solution to form the nanofiber scaffold and delivers the raw material solution to the spinning nozzle through the material tube. Under an appropriate environment temperature and an external electric field, the spinning nozzle ejects the filamentous raw material solution toward the material collection plate, thereby forming nanofibers while the environment temperature is controlled by the temperature controller and the external electric field is provided by the electrostatic generator.

In Step 12 of the electrospinning process, configuring the operating conditions of the electrospinning apparatus includes setting the environment temperature, a working voltage, feed rate, a rotation speed of the collection plate, and a distance between the spinning nozzle and the collection plate. In an embodiment, the environment temperature is set between 40 ℃ and 60 ℃, the working voltage is set between 20 kV and 100 kV, the feed rate is set between 1 ml/hr and 100 ml/hr, the rotation speed is set between 3 cm/min and 50 cm/min, and the distance between the spinning nozzle and the collection plate is between 3 cm and 20 cm, though the invention is not limited thereto.

Step S14 of the drying process includes removing water from the nanofiber scaffolds by freezing the nanofiber scaffolds, thereby forming the artificial bone material. In one embodiment, the nanofiber scaffolds formed by the electrospinning process are placed in a freezer under a freezing temperature of -196 ℃ to 4 ℃ for a time period of 24 hours to 72 hours. However, the invention is not limited thereto.

Gelatin mixture and the artificial bone material of the present invention are separately co-cultured with mesenchymal stem cells (MSCs). Here, the group in which the gelatin mixture is co-cultured with the MSCs is used as a control group, and the group in which the artificial bone material of the present invention is co-cultured with the MSCs is used as the operation group. On days 7, 14, 21, and 28 of co-culture, the MSCs from both the control group and the experimental group are collected and subjected to quantitative polymerase chain reaction (qPCR) analysis to detect the expression of osteogenic differentiation marker genes, in order to evaluate the efficacy of the artificial bone material on the induction of the MSCs. The osteogenic differentiation marker genes include ALP, BGLAP, COL1A1, RUNX2, SP7, and SPARC genes.

The ALP (alkaline phosphatase) gene plays a critical role in osteogenic differentiation and is closely associated with the mineralization process of the bone tissue. The ALP gene promotes the maturation of osteoblasts by hydrolyzing organic phosphates and releasing inorganic phosphates, thereby participating in the mineralization process and promoting the deposition of the bone minerals. The BGLAP (bone gamma-carboxyglutamate protein, also known as osteocalcin) gene encodes a non-collagenous protein secreted by osteoblasts and is intimately involved in bone mineralization and remodeling. During osteogenic differentiation, the BGLAP gene regulates the expression of osteocalcin, which binds to hydroxyapatite and facilitates calcium deposition in the bone matrix, thus enhancing bone mineralization. The COL1A1 (collagen type I alpha 1 chain) gene encodes the α1 chain of type I collagen, which is the main component of the bone matrix, accounting for approximately 90% of the organic portion of the bone matrix. During osteogenic differentiation, osteoblasts produce large amounts of type I collagen, which serves as a scaffold for mineral deposition, including hydroxyapatite, thereby supporting bone formation and hardening. The RUNX2 gene (Runt related transcription factor 2) is an essential transcription factor for osteoblast differentiation, particularly during the early stage of bone formation. The RUNX2 gene is a main regulator of bone-specific genes, such as COL1A1, BGLAP (osteocalcin), and ALPL, which encode proteins related to bone formation and mineralization. The SP7 gene (Osterix) may regulate the expression of multiple osteogenesis-related genes, including COL1A1 and BGLAP, both of which encode major components of the bone matrix. As such, SP7 is crucial for the maturation of osteoblasts. The SPARC gene (secreted protein acidic and rich in cysteine) is a secreted protein that influences osteoblast behavior by regulating the composition and structure of the extracellular matrix, thereby promoting osteogenic differentiation. During bone formation, the SPARC gene supports the development of the bone matrix by facilitating mineral deposition. It may bind to hydroxyapatite and helps osteoblasts regulate their surrounding microenvironment, enhancing their differentiation potential. In addition, the SPARC gene also participates in the regulation of cell-cell interactions related to bone remodeling.

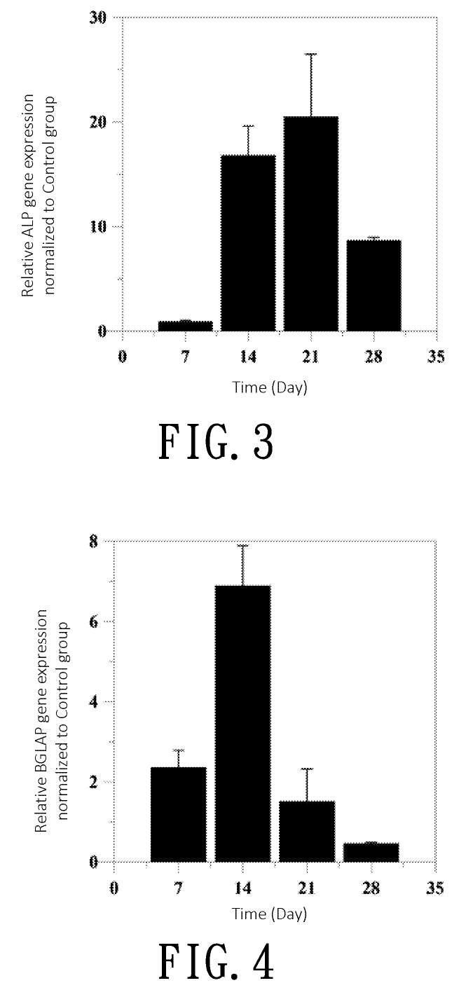

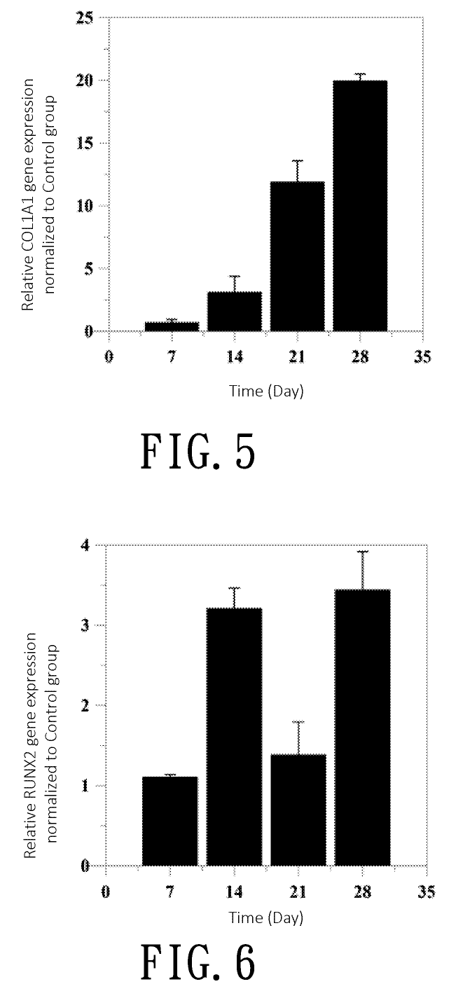

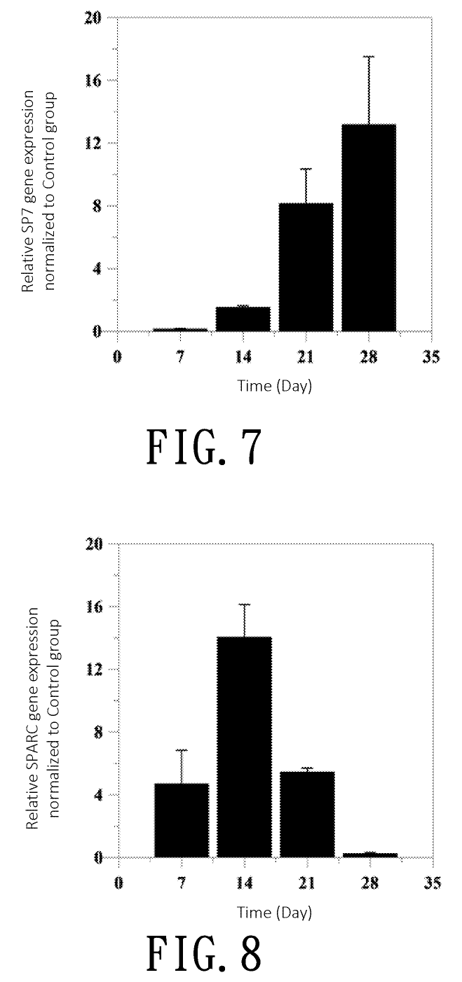

In a first embodiment, the artificial bone material and MSCs are co-cultured and regarded as an operation group 1. The experimental results of the operation group 1 are shown as FIG. 3 to FIG. 8. FIG. 3 is a schematic diagram showing normalized expression amounts of the ALP gene in the artificial bone material according to the first embodiment of the invention, relative to a control group. FIG. 4 is a schematic diagram showing normalized expression amounts of the BGLAP gene in the artificial bone material according to the first embodiment of the invention, relative to the control group. FIG. 5 is a schematic diagram showing normalized expression amounts of the COL1A1 gene in the artificial bone material according to the first embodiment of the invention, relative to the control group. FIG. 6 is a schematic diagram showing normalized expression amounts of the RUNX2 gene in the artificial bone material according to the first embodiment of the invention, relative to the control group. FIG. 7 is a schematic diagram showing normalized expression amounts of the SP7 gene in the artificial bone material according to the first embodiment of the invention, relative to the control group. FIG. 8 is a schematic diagram showing normalized expression amounts of the SPARC gene in the artificial bone material according to the first embodiment of the invention, relative to the control group. In the control group, a gelatin-based mixture, including 91 wt% gelatin and 9 wt% hydroxyapatite, is used. In the operation group 1 of the first embodiment, the artificial bone material includes, for example, 90.89 wt% gelatin, 9.09 wt% hydroxyapatite, and 0.02 wt% Pueraria lobata. As shown in FIG. 3 to FIG. 8, the expression amounts of the osteogenic marker genes ALP, BGLAP, COL1A1, RUNX2, SP7, and SPARC in MSCs significantly increase in the operation group 1 in comparison with the control group. These results indicate that the artificial bone material of the operation group 1 effectively promotes the expression amounts of the osteogenic differentiation of the MSCs relative to the control group.

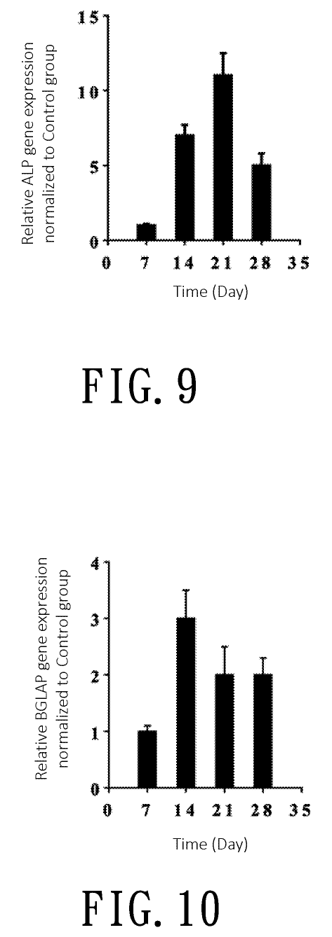

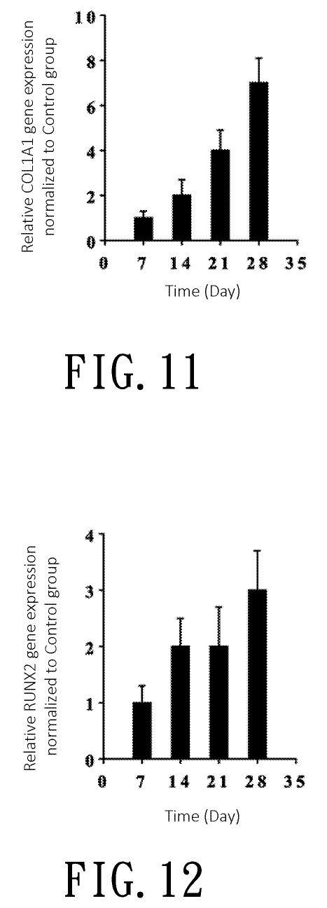

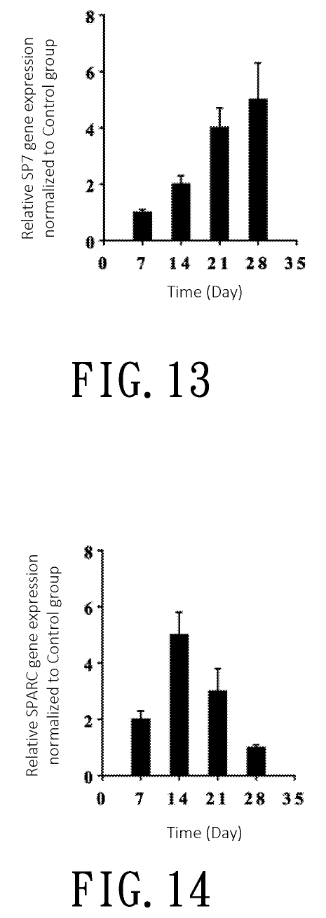

In a second embodiment, the artificial bone material and MSCs are co-cultured to form an operation group 2. The experimental results of the operation group 2 are shown as FIG. 9 to FIG. 14. FIG. 9 is a schematic diagram showing normalized expression amounts of the ALP gene in the artificial bone material according to the second embodiment of the invention, relative to a control group. FIG. 10 is a schematic diagram showing normalized expression amounts of the BGLAP gene in the artificial bone material according to the second embodiment of the invention, relative to the control group. FIG. 11 is a schematic diagram showing normalized expression amounts of the COL1A1 gene in the artificial bone material according to the second embodiment of the invention, relative to the control group. FIG. 12 is a schematic diagram showing normalized expression amounts of the RUNX2 gene in the artificial bone material according to the second embodiment of the invention, relative to the control group. FIG. 13 is a schematic diagram showing normalized expression amounts of the SP7 gene in the artificial bone material according to the second embodiment of the invention, relative to the control group. FIG. 14 is a schematic diagram showing normalized expression amounts of the SPARC gene in the artificial bone material according to the second embodiment of the invention, relative to the control group. In the control group, a gelatin-based mixture, including 91 wt% gelatin and 9 wt% hydroxyapatite, is used. In the operation group 2 of the second embodiment, the artificial bone material includes, for example, 92.59 wt% gelatin, 6.17 wt% hydroxyapatite, and 1.23 wt% Pueraria lobata. As shown in FIG. 9 to FIG. 14, the expression amounts of the osteogenic marker genes ALP, BGLAP, COL1A1, RUNX2, SP7, and SPARC in MSCs significantly increase in the operation group 2 in comparison with the control group. These results indicate that the artificial bone material of the operation group 2 effectively promotes the expression amounts of the osteogenic differentiation of the MSCs relative to the control group.

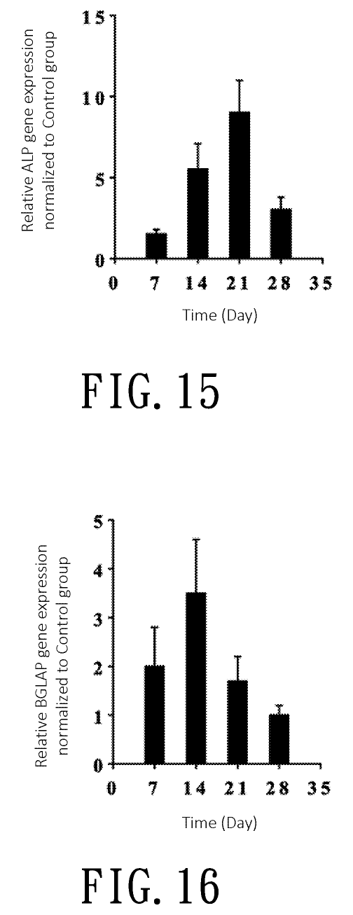

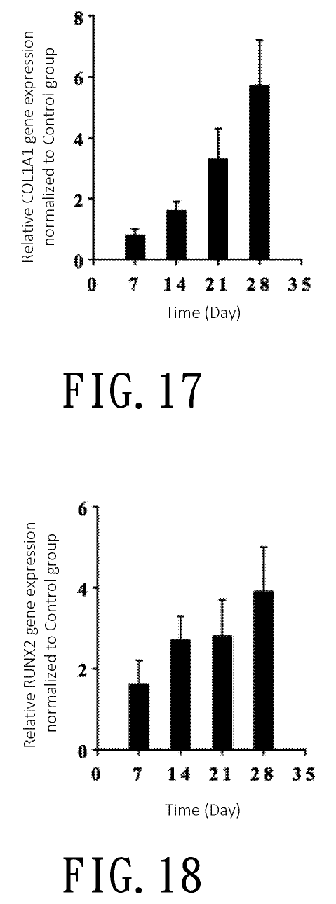

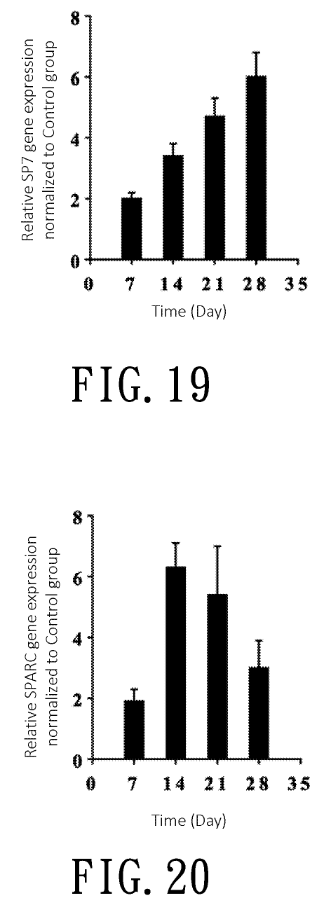

In a third embodiment, the artificial bone material and MSCs are co-cultured to form an operation group 3. The experimental results of the operation group 3 are shown as FIG. 15 to FIG. 20. FIG. 15 is a schematic diagram showing normalized expression amounts of the ALP gene in the artificial bone material according to the third embodiment of the invention, relative to a control group. FIG. 16 is a schematic diagram showing normalized expression amounts of the BGLAP gene in the artificial bone material according to the third embodiment of the invention, relative to the control group. FIG. 17 is a schematic diagram showing normalized expression amounts of the COL1A1 gene in the artificial bone material according to the third embodiment of the invention, relative to the control group. FIG. 18 is a schematic diagram showing normalized expression amounts of the RUNX2 gene in the artificial bone material according to the third embodiment of the invention, relative to the control group. FIG. 19 is a schematic diagram showing normalized expression amounts of the SP7 gene in the artificial bone material according to the third embodiment of the invention, relative to the control group. FIG. 20 is a schematic diagram showing normalized expression amounts of the SPARC gene in the artificial bone material according to the third embodiment of the invention, relative to the control group. In the control group, a gelatin-based mixture, including 91 wt% gelatin and 9 wt% hydroxyapatite, is used. In the operation group 2 of the third embodiment, the artificial bone material includes, for example, 48.78 wt% gelatin, 2.44 wt% hydroxyapatite, and 48.78 wt% Pueraria lobata. As shown in FIG. 15 to FIG. 20, the expression amounts of the osteogenic marker genes ALP, BGLAP, COL1A1, RUNX2, SP7, and SPARC in MSCs significantly increase in the operation group 3 in comparison with the control group. These results indicate that the artificial bone material of the operation group 3 effectively promotes the expression amounts of the osteogenic differentiation of the MSCs relative to the control group.

The artificial bone material of the present invention effectively promotes the differentiation of mesenchymal stem cells and facilitates bone regeneration for the repair of bone defects through a combination of the natural polymer, the inorganic salt, and the Pueraria lobata. In addition, the use of electrospinning technology enhances the mechanical properties of the artificial bone material.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An artificial bone material, comprising:
a natural polymer;
an inorganic salt; and
Pueraria lobata;
wherein the natural polymer comprises at least one of gelatin, collagen, hyaluronic acid, alginate, chitosan, and their derivatives, and the inorganic salt comprises at least one of hydroxyapatite, tricalcium phosphate, dicalcium phosphate, dicalcium phosphate dihydrate, tetracalcium phosphate, and their derivatives.

2. The artificial bone material according to claim 1, wherein, based on a total weight of the artificial bone material, a weight percentage of the natural polymer is between 45 wt% and 95 wt%, a weight percentage of the inorganic salt is between 1 wt% and 10 wt%, and a weight percentage of Pueraria lobata is between 0.01 wt% and 50 wt%.

3. A method for preparing an artificial bone material, the method comprising:
preparing a bone material solution by mixing a natural polymer, an inorganic salt, and Pueraria lobata in sterile water to form the bone material solution;
performing an electrospinning process on the bone material solution to form a nanofiber scaffold; and
performing a drying process to freeze-dry the nanofiber scaffold to remove water and form the artificial bone material.

4. The method according to claim 3, wherein the bone material solution comprises the natural polymer at a concentration of between 0.1 g/L and 1000 g/L, the inorganic salt at a concentration of between 0.1 g/L and 1000 g/L, and the Pueraria lobata at molar concentration of between 1 μM and 5 M.

5. The method according to claim 4, wherein the bone material solution comprises the natural polymer at a concentration of between 1 g/L and 500 g/L, the inorganic salt at a concentration of between 0.5 g/L and 100 g/L, and the Pueraria lobata at molar concentration of between 10 μM and 5 M.

6. The method according to claim 3, wherein preparing the bone material solution comprises:
dissolving the natural polymer into the sterile water to form a natural polymer solution;
adding the inorganic salt into the natural polymer solution to obtain a mixed solution; and
adding the Pueraria lobata into the mixed solution to form the bone material solution;
wherein temperatures of the natural polymer solution, the mixed solution, and the bone material solution are between 35 ℃ and 65 ℃.

7. The method according to claim 3, wherein the natural polymer comprises at least one of gelatin, collagen, hyaluronic acid, alginate, chitosan, and their derivatives, and the inorganic salt comprises at least one of hydroxyapatite, tricalcium phosphate, dicalcium phosphate, dicalcium phosphate dihydrate, tetracalcium phosphate, and their derivatives.

8. The method according to claim 3, wherein the electrospinning process is operated at an ambient temperature of 35 ℃ to 65 ℃ and at a working voltage of 10 kV to 100 kV.

9. The method according to claim 3, wherein the drying process is performed at a freezing temperature of -196 ℃ to 4 ℃ for a duration of 24 hours to 72 hours.
